(19) United States
(12) Patent Application Publication
(10) Pub. No.: US 2025/0090104 A1
(43) Pub. Date: Mar. 20, 2025

(54) Method of Predicting the Likelihood of Hyperglycemia

(71) Applicants: National University of Singapore, Singapore (SG); Singapore Health Services Pte Ltd, Singapore (SG)

(72) Inventors: Xinke Li, Singapore (SG); Yeow Meng Chee, Singapore (SG); Ngeow Khing Chia, Singapore (SG); Su Yen Goh, Singapore (SG); Ann Kerwen Kwee, Singapore (SG)

(21) Appl. No.: 18/568,617

(22) PCT Filed: Jun. 9, 2022
(86) PCT No.: PCT/SG2022/050395

§ 371 Date: Dec. 8, 2023

(30) Foreign Application Priority Data
Jun. 9, 2021 (SG) 10202106153U

(51) Int. Cl. A61B 5/00; A61B 5/08; G01N 33/497

(52) U.S. Cl. CPC A61B 5/7275; A61B 5/082; A61B 5/7264; A61B 5/7282; G01N 33/4972; G01N 33/4975; G01N 2800/042; G01N 2800/50

(57) ABSTRACT

The present disclosure relates to a method and kits for detecting or determining the presence or likelihood of hyperglycemia in a subject. More particularly, the present invention relates to methods and kits for detecting or determining the likelihood of hyperglycemia in a subject based on a set of biomarkers in a breath sample, the biomarkers comprise at least one of cymene, butanol and pentanol.

### FIELD OF INVENTION

The present disclosure relates to a method and kits for detecting or determining the presence or likelihood of hyperglycemia in a subject. More particularly, the present invention relates to methods and kits for detecting or determining the likelihood of hyperglycemia in a subject based on a set of biomarkers in a breath sample.

### BACKGROUND

Diabetes is a disease of major concern both globally and regionally. It is a leading cause of death in most countries. In 2013, the International Diabetes Federation (IDF) estimated that; 382 million people have diabetes worldwide, and by 2035, this is predicted to rise to 592 million. Eighty percent live in low- and middle-income countries, and of the total, more than 60% live in Asia, with almost one-third in China. Major increases in the prevalence of diabetes have occurred in developing countries due to rapid and ongoing socioeconomic transition and will likely lead to further rises. The prevalence of both type 1 diabetes (T1DM) and type 2 diabetes (T2DM) has increased significantly over the recent decades. T2DM is much more common and has been the main driver for the increase in global diabetes prevalence. Therefore, it will be the focus of this review. It should be noted that with regard to diabetes prevalence, only broad comparisons can be made among studies on which national and global estimates by the IDF are based. This is because there are marked differences in age-groups, survey methodologies, diagnostic criteria, and other aspects of these studies in the various countries in Asia and the Pacific. Despite this, almost all developing countries in the Western Pacific region (WPR) and in South Asia have shown escalating rates.

Diabetes has become a great threat to human health. The timely diagnosis and frequent monitoring are important tenets for managing the disease. In order to avoid long term complications of T2DM, capillary blood glucose levels (BGLs) have to be kept within a tight range. Capillary BGLs are drawn by means of finger-prick and have to be done frequently in order to decrease blood glucose variability. Despite the accuracy of this method, it is also painful, invasive, and inconvenient. Frequent blood testing is necessary for patients undergoing treatment. American Diabetes Association recommends to self-monitor blood glucose concentrations 3+ times daily via finger sticks especially if the patient is on insulin therapy. Therefore, non-invasive diabetes screening and BGL prediction has been gathering more interest of late. Approaches include reverse iontophoresis, fluorescence technology, bio-impedance spectroscopy, and so on have been studied. These approaches are painless and convenient, but there are still disadvantages such as lack of specificity, inaccuracy due to subject's movement and sweating, skin irritation, etc.

Patients with poorly controlled DM lead to an immunocompromised state because of the consistent hyperglycemia. This puts patients at higher risks of complications and also susceptible to other infections which leads to inflammation. Circulating levels of interleukin-6 (IL-6) and tumor necrosis factor-α (TNF-α) are elevated in diabetic patients. Diabetes can be detected through a blood glucose test and correlated to their breath VOCs. There are various methods to diagnose T2DM such as a random blood glucose reading (>11.1 mmol/L), a fasting blood glucose reading (>7 mmol/L) or haemoglobin A1c (Hba1c) (>48 mmol/mol or >6.5%). However, there are a few issues fraught with using Hba1c for diagnosis of T2DM. A normal A1C level is below 5.7%, a level of 5.7% to 6.4% indicates prediabetes, and a level of 6.5% or more indicates diabetes. However, a value of less than 48 mmol/mol (6.5%) does not exclude diabetes diagnosed using glucose tests. Within the 5.7% to 6.4% prediabetes range, the higher the A1C, the greater risk is for patients to develop type 2 diabetes. A blood sugar level less than 140 mg/dl (7.8 mmol/L) is considered normal. A blood sugar level from 140 to 199 mg/dL (7.8 to 11.0 mmol/L) is considered prediabetes. This is sometimes referred to as impaired glucose tolerance. A blood sugar level of 200 mg/dL (11.1 mmol/L) or higher indicates type 2 diabetes.

Hemoglobin is a protein found in red blood cells. It gives blood its red color, and its job is to carry oxygen throughout the body. The sugar in blood is called glucose. When glucose builds up in the blood, it binds to the hemoglobin in red blood cells. The Alc test measures how much glucose is bound. The hemoglobin Alc test measures the subjects average level of blood sugar over the past 2 to 3 months. It's also called HbA1c, glycated hemoglobin test, and glycohemoglobin. A 1 day's test results do not give the subject the complete picture of how their treatment is working. Subjects who have diabetes need this test regularly to see if their levels are staying within range. It can tell if the subject need to adjust diabetes medicines. The Alc test is also used to diagnose diabetes. People with diseases affecting hemoglobin, such as anemia, may get misleading results with this test. Other things that can affect the results of the hemoglobin Alc include supplements such as vitamins C and E and high cholesterol levels. Kidney disease and liver disease may also affect the test.

While large resources have been invested worldwide in developing non-invasive devices for diabetes management, progress has been slow. Two early patents for non-invasive glucose testing, based on the changes of light through the eye, were filed in 1974 (U.S. Pat. Nos. 3,958,560 and 3,963,019). However, no FDA-approved products are commercially available nearly 40 years later. In fact, some devices for non-invasive blood glucose testing have even been withdrawn from the market. Measuring plasma insulin is clinically useful to assess pre-diabetic states (as an indicator of impaired glucose metabolism, it increases earlier than glycemia itself), to stage the progression of T2DM from initial insulin resistance to eventual pancreatic failure, and to differentiate the increasingly common states in which components of both T1DM and T2DM are simultaneously present. Monitoring insulin can also give insight to other aspects of metabolism; insulin not only regulates glucose disposal but also exerts a strong anti-lipolytic effect, which is markedly reduced in patients with insulin resistance. However, tests for insulin concentrations and sensitivity are unfortunately very laborious. Similarly, testing circulating lipids is important for diabetic patients because hyperlipidemia is an independent risk factor for heart disease. As lipids increase ketone body formation, their alterations may also be associated with changes in insulin or glucose metabolism. Knowing the interplay of these metabolic variables may allow clinicians to have a more comprehensive insight into their patients' health, and reliable non-invasive monitoring would certainly improve diagnosis, and treatment of diabetes. Real-time insulin or lipid meters (comparable to glucose meters) do not yet exist.

Accordingly, it is generally desirable to overcome or ameliorate one or more of the above-mentioned difficulties.

### SUMMARY

Disclosed herein is a method of predicting the likelihood of hyperglycemia in a subject, the method comprising determining the level of one or more biomarkers in a sample obtained from the subject, wherein the level or levels of the one or more biomarkers, or a value derived therefrom, as compared to a reference predicts the likelihood of hyperglycemia in the subject; and wherein the one or more biomarkers comprises at least one of cymene, butanol and pentanol.

Disclosed herein is a method of treating hyperglycemia in a subject, the method comprising: a) determining the level of one or more biomarkers in a sample obtained from the subject, wherein the level or levels of the one or more biomarkers, or a value derived therefrom, as compared to a reference predicts the likelihood of hyperglycemia in the subject; wherein the one or more biomarkers comprises at least one of cymene, butanol and pentanol, and b) exposing the subject to a treatment regimen to treat hyperglycemia in the subject.

Disclosed herein is a method of treating hyperglycemia in a subject, the method comprising: a) selecting a subject who is likely to have hyperglycemia, wherein the level or levels of the one or more biomarkers, or a value derived therefrom, in a sample obtained from the subject as compared to a reference predicts that the subject is likely to have hyperglycemia, wherein the one or more biomarkers comprises at least one of cymene, butanol and pentanol, and b) exposing the subject to a treatment regimen to treat hyperglycemia in the subject.

Disclosed herein is a method of monitoring blood glucose level in a subject, the method comprising determining the level of one or more biomarkers in a sample obtained from the subject, wherein the level or levels of the one or more biomarkers, or a value derived therefrom, as compared to a reference provides an indication of the blood glucose level in the subject; and wherein the one or more biomarkers comprises at least one of cymene, butanol and pentanol.

Disclosed herein is a method of identifying a subject who is at risk of developing diabetes or a diabetic condition, the method comprising determining the level of one or more biomarkers in a sample obtained from the subject, wherein the level or levels of the one or more biomarkers, or a value derived therefrom, as compared to a reference identifies a subject as one who is at risk of developing diabetes or a diabetic condition; and wherein the one or more biomarkers comprises at least one of cymene, butanol and pentanol.

Disclosed herein is a method of preventing or delaying the onset of diabetes or a diabetic condition in a subject, the method comprising a) determining the level of one or more biomarkers in a sample obtained from the subject, wherein the level or levels of the one or more biomarkers, or a value derived therefrom, as compared to a reference identifies a subject as one who is at risk of developing diabetes or a diabetic condition; wherein the one or more biomarkers comprises at least one of cymene, butanol and pentanol; and b) exposing the subject to a treatment regimen for preventing or delaying the onset of the diabetes or diabetic condition.

### BRIEF DESCRIPTION OF DRAWINGS

Embodiments of the present invention are hereafter described, by way of non-limiting example only, with reference to the accompanying drawings in which:

FIG. 1. Diagrams of Random Forest

FIG. 2: A decision tree for predicting the likelihood of hyperglycemia.

### DETAILED DESCRIPTION

The present specification teaches a method of predicting the likelihood of hyperglycemia in a subject.

Disclosed herein is a method of predicting the likelihood of hyperglycemia in a subject, the method comprising determining the level of one or more biomarkers in a sample obtained from the subject, wherein the level or levels of the one or more biomarkers, or a value derived therefrom, as compared to a reference predicts the likelihood of hyperglycemia in the subject; and wherein the one or more biomarkers comprises at least one of cymene, butanol and pentanol.

The method as referred to herein may comprise determining the levels of cymene, butanol and/or pentanol in the sample. For example, it may comprise determining the level(s) of a) cymene, b) butanol, c) pentanol, d) cymene and butanol, e) cymene and pentanol, f) butanol and pentanol or g) cymene, butanol and pentanol.

The method as referred to herein may comprise determining the level of at least one of cymene, butanol, pentanol and acetone. The method as referred to herein may comprise determining the levels of cymene, butanol, pentanol and acetone in the sample.

The method may further comprises determining the level of at least one of acetone, ethanol, Propane, Carbon monoxide, Ethyl benzene, Xylene, methanol and Isoprene.

The method may further comprises determining the level of at least one of Methyl Nitrate, Pentyl nitrate, Toulene, Tridecane, Undecane, Trimethyldecane and Pentane.

In one embodiment, the method comprises determining the level of one or more biomarkers listed in Table 1. The method may also comprise determining the level of a panel of biomarkers as shown in Table 2.

TABLE 1
Biomarker list

| | Detection | | |
|---|---|---|---|
| | Decision | | |
| | Priority | Biomarkers | Pathway or known possibly |
| No. | Level | (VOCs) | relation |
| 1 | Level 1 | cymene (such as | Cytokine & Pathogenic |
| | | p-cymene) | processes |
| 2 | Level 1 | butanol (such as | Cytokine & Pathogenic |
| | | 1-butanol) | processes |
| 3 | Level 1 | pentanol (such as | Cytokine & Pathogenic |
| | | 1-pentanol) | processes |
| 4 | Level 1 | Acetone* | Glycolysis/Pyruvate |
| | | | Metabolism |
| 1 | Level 2 | ethanol | Glycolysis/Gluconeogenesis |
| 2 | Level 2 | Propane | N-4 fatty acid peroxidation |
| | | | protein oxidation |
| 3 | Level 2 | Carbon monoxide | |
| 4 | Level 2 | Ethyl benzene | |
| 5 | Level 2 | Xylene (such as M, | |
| | | P and/or O xylene) | |
| 6 | Level 2 | methanol | |
| 7 | Level 2 | Isoprene | |
| 1 | Level 3 | Methyl Nitrate | |
| 2 | Level 3 | Pentyl nitrate (such | |
| | | as 2-pentyl nitrate) | |
| 3 | Level 3 | Toulene | |
| 4 | Level 3 | Tridecane | |
| 5 | Level 3 | Undecane | |
| 6 | Level 3 | Trimethyldecane | |
| | | (such as | |
| | | Trimethyldecane | |
| | | (2,7,8) and/or | |
| | | Trimethyldecane | |
| | | (3,5,7)) | |
| 7 | Level 3 | Pentane | |

*The exhaled acetone at M/z 59.05 measured in the range of ~200-1000 ppb for healthy humans and over 1800 ppb for elevated glucose level.

TABLE 2
List of Potential Biomarker Combinations

| | Biomarkers |
|---|---|
| 1 | A panel comprising at least one of cymene, butanol, pentanol, acetone, ethanol, propane, carbon |
| | monoxide, ethyl benzene, Xylene, methanol, isoprene, methyl nitrate, Pentyl nitrate, toluene, |
| | tridecane, undercane, Trimethyldecane and Pentane. |
| 2 | The panel according to 1, wherein the panel comprises cymene. |
| 3 | The panel according to 1 or 2, wherein the panel comprises butanol. |
| 4 | The panel according to any one of 1 to 3, wherein the panel comprises pentanol. |
| 5 | The panel according to any one of 1 to 4, wherein the panel comprises acetone. |
| 6 | The panel according to any one of 1 to 5, wherein the panel comprises ethanol. |
| 7 | The panel according to any one of 1 to 6, wherein the panel comprises propane. |
| 8 | The panel according to any one of 1 to 7, wherein the panel comprises carbon monoxide. |
| 9 | The panel according to any one of 1 to 8, wherein the panel comprises ethyl benzene. |
| 10 | The panel according to any one of 1 to 9, wherein the panel comprises Xylene (such as M/P |
| | and/or O-Xylene). |
| 11 | The panel according to any one of 1 to 10, wherein the panel comprises methanol. |
| 12 | The panel according to any one of 1 to 11, wherein the panel comprises isoprene. |
| 13 | The panel according to any one of 1 to 12, wherein the panel comprises methyl nitrate. |
| 14 | The panel according to any one of 1 to 13, wherein the panel comprises Pentyl nitrate (such as |
| | 2-pentyl nitrate). |
| 15 | The panel according to any one of 1 to 14, wherein the panel comprises toluene. |
| 16 | The panel according to any one of 1 to 15, wherein the panel comprises tridecane. |
| 17 | The panel according to any one of 1 to 16, wherein the panel comprises undercane. |
| 18 | The panel according to any one of 1 to 17, wherein the panel comprises Trimethyldecane (such |
| | as Trimethyldecane (2,7,8) and/or Trimethyldecane (3,5,7)). |
| 19 | The panel according to any one of 1 to 18, wherein the panel comprises pentane. |
| 20 | The panel according to any one of 1 to 19, wherein the panel comprises one biomarker. |
| 21 | The panel according to any one of 1 to 19, wherein the panel comprises two biomarkers. |
| 22 | The panel according to any one of 1 to 19, wherein the panel comprises three biomarkers. |
| 23 | The panel according to any one of 1 to 19, wherein the panel comprises four biomarkers. |
| 24 | The panel according to any one of 1 to 19, wherein the panel comprises five biomarkers. |
| 25 | The panel according to any one of 1 to 19, wherein the panel comprises six biomarkers. |
| 26 | The panel according to any one of 1 to 19, wherein the panel comprises seven biomarkers. |
| 27 | The panel according to any one of 1 to 19, wherein the panel comprises eight biomarkers. |
| 28 | The panel according to any one of 1 to 19, wherein the panel comprises nine biomarkers. |
| 29 | The panel according to any one of 1 to 19, wherein the panel comprises ten biomarkers. |
| 30 | The panel according to any one of 1 to 19, wherein the panel comprises eleven biomarkers. |
| 31 | The panel according to any one of 1 to 19, wherein the panel comprises twelve biomarkers. |
| 32 | The panel according to any one of 1 to 19, wherein the panel comprises thirteen biomarkers. |
| 33 | The panel according to any one of 1 to 19, wherein the panel comprises fourteen biomarkers. |
| 34 | The panel according to any one of 1 to 19, wherein the panel comprises fifteen biomarkers. |
| 35 | The panel according to any one of 1 to 19, wherein the panel comprises sixteen biomarkers. |
| 36 | The panel according to any one of 1 to 19, wherein the panel comprises seventeen biomarkers. |
| 37 | The panel according to any one of 1 to 19, wherein the panel comprises eighteen biomarkers. |
| 38 | The panel according to any one of 1 to 19, wherein the panel comprises nineteen biomarkers. |
| 39 | The panel according to any one of 1 to 19, wherein the panel comprises twenty biomarkers or |
| | more. |

The method as referred to herein may comprise determining the levels of 1, 2, 3, 4, 5, 6, 7, 8, 9, 10, 11, 12, 13, 14, 15, 16, 17. 18, 19, 20 or more biomarkers.

In one embodiment, a biomarker as defined herein can be an isomer of the biomarker and includes any and/or possible structural or geometric isomers and stereoisomers of the biomarker. The term “isomer” as used herein includes any and/or all structural isomers, geometric isomers and stereoisomers (e.g., enantiomers, diasteromers, etc.). For example, “isomer” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the biomarker is made up of a significantly greater proportion of one enantiomer. In certain embodiments the biomarker of the present invention is made up of at least about 50%, 60%, 70%, 80%, 90%, 95%, 95% or 99% by weight of a preferred isomer In other embodiments the biomarker is made up of at least about 50%, 60%, 70%, 80%, 90%, 95%, 98%, or 99% by weight of a preferred enantiomer. For instance, an “isomer” of cymene can include p-cymene, o-cymene or o-cymene.

As used herein the terms “level” and “amount” are used interchangeably to refer to a quantitative amount (e.g., weight or moles or number), a semi-quantitative amount, a relative amount (e.g., weight % or mole % within class or a ratio), a concentration, and the like. Thus, in reference to the amount or level of a biomarker, the terms encompasses absolute or relative amounts or concentrations of biomarkers in a sample, including ratios of levels of biomarkers, and odds ratios of levels or ratios of odds ratios. Levels or amounts may also be reflective of an individual subject or of cohorts of subjects, the latter being expressed, for example, as mean or medium levels.

As used herein, the term “sample” includes tissues, cells, body fluids and isolates thereof etc., isolated from a subject, as well as tissues, cells and fluids etc. present within a subject (i.e. the sample is in vivo). Examples of samples include: whole blood, blood fluids (e.g. serum and plasm), lymph and cystic fluids, saliva, sputum, stool, tears, mucus, hair, skin, breath (e.g. exhaled breath), ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

In one embodiment, the sample is a breath sample. The method as defined herein may comprise obtaining exhaled breath from a subject. In one embodiment, the sample is exhaled breath. In one embodiment, the sample is end-tidal breath.

The term “biomarker” refers to a measurable characteristic that reflects the presence or nature (e.g., severity) of a physiological and/or pathophysiological state. It can be an indicator of the risk of having or developing a particular physiological or pathophysiological state.

In one embodiment, the biomarker is a Volatile Organic Compound (VOC). For example, VOCs from the breath of a subject may be collected in a sample, e.g., on a filter, either directly or indirectly. The breath samples may be collected in a collection device which may include sorbent tubes, tedlar bags, canisters etc. It can also comprise collecting samples through a real-time breath sampler. In some embodiments, the breath sample is directly obtained from a subject at or near the laboratory or location where the biological sample will be analyzed. In other embodiments, the breath sample is obtained by a third party and then transferred, e.g., to a separate entity or location for analysis.

In other embodiments, the sample is obtained and tested in the same location using a point-of care test. In these embodiments, said obtaining refers to receiving the sample, e.g., from the patient, from a laboratory, from a doctor's office, from the mail, courier, or post office, etc. The method may further comprise reporting the determination or test results to the subject, a health care payer, an attending clinician, a pharmacist, a pharmacy benefits manager, or any person that the determination or test results may be of interest. The detection of the volatile organic compounds as referred to herein may be detecting using an analytical instrument. Examples of analytical instruments include GC-MS (gas chromatography mass spectrometry), PTR-MS (proton transfer reaction mass spectrometry), SIFT-MS (selected ion flow tube mass spectrometry), sensor technologies.

In one embodiment, a change in the level or levels of the one or more biomarkers, or a value derived therefrom, as compared to a reference predicts the likelihood of hyperglycemia in the subject. The change may be an increase or a decrease.

As used herein, the term “increase” or “increased’ with reference to a biomarker refers to a statistically significant and measurable increase in the biomarker as compared to a reference or control. The increase is preferably an increase of at least about 10%, or an increase of at least about 20%, or an increase of at least about 30%, or an increase of at least about 40%, or an increase of at least about 50%.

In one embodiment, an increased level of each of the biomarker as compared to a reference or control predicting the likelihood of hyperglycemia in a subject. The increase in level may be an increase of 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 fold, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times or 100 times or anywhere in between as compared to a reference or control.

As used herein, the term “decrease” or “decreased’ with reference to a biomarker refers to a statistically significant and measurable decrease in the biomarker as compared to a reference or control. The decrease is preferably a decrease of at least about 10%, or a decrease of at least about 20%, or a decrease of at least about 30%, or a decrease of at least about 40%, or a decrease of at least about 50%. The decrease in level may refer to a biomarker having 0.9 times or less, 0.8 times or less, 0.7 times or less, 0.6 times or less, 0.5 times or less, 0.4 times or less, 0.3 times or less, 0.2 times or less, 0.1 times or less or anywhere in between as compared to the level of a control.

By “about” is meant a quantity, level, value, number, frequency, percentage, dimension, size, amount, weight, position or length that varies by as much as 10, 9, 8, 7, 6, 5, 4, 3, 2 or 1% to a reference quantity, level, value, number, frequency, percentage, dimension, size, amount, weight, position or length. When the term “about” is used in conjunction with a numerical range, it modifies that range by extending the boundaries above and below the numerical values set forth. In general, the term “about” is used herein to modify a numerical value above and below the stated value by a variance of as much as 10, 9, 8, 7, 6, 5, 4, 3, 2 or 1%

In one embodiment, the reference or control is a sample obtained from a subject who is healthy. The reference or control may comprise one or more reference biomarkers for comparison with the levels of the one or more biomarkers in the sample. The reference or control may also be samples obtained from a group of subjects who are healthy. Each subject may be one who does not have hyperglycemia and/or diabetes.

As used herein the term “hyperglycemia” refers to an excess of glucose in the bloodstream. It can occur in a variety of diseases due to insufficient insulin in the bloodstream and/or due to excessive intake of simple carbohydrates.

In one embodiment, a healthy subject or a subject with no hyperglycemia has about 0.02 ppb to about 0.6 ppb cymene. In one embodiment, a healthy subject or a subject with no hyperglycemia has about 0.5 ppb to about 5 ppb butanol. In one embodiment, a healthy subject or a subject with no hyperglycemia has about 0.1 ppb to about 2.1 ppb pentanol.

In one embodiment, a subject who is likely to have hyperglycemia has at least about 0.6 ppb, at least about 0.7 ppb, at least about 0.8 ppb, at least about 0.9 ppb, at least about 1 ppb, at least about 2 ppb, at least about 3 ppb, at least about 4 ppb, at least about 5 ppb, at least about 6 ppb, at least about 7 ppb, at least about 8 ppb, at least about 9 ppb, at least about 10 ppb, at least about 20 ppb, at least about 30 ppb, at least about 40 ppb, at least about 50 ppb, at least about 60 ppb, at least about 70 ppb, at least about 80 ppb, at least about 90 ppb, at least about 100 ppb or anywhere in between of cymene.

In one embodiment, a subject who is likely to have hyperglycemia has at least about 6 ppb, at least about 6.1 ppb, at least about 6.2 ppb, at least about 6.3 ppb, at least about 6.4 ppb, at least about 6.4 ppb, at least about 6.5 ppb, at least about 6.6 ppb, at least about 6.7 ppb, at least about 6.8 ppb at least about 6.9 ppb, at least about 7 ppb, at least about 8 ppb, at least about 9 ppb, at least about 10 ppb, at least about 20 ppb, at least about 30 ppb, at least about 40 ppb, at least about 50 ppb, at least about 60 ppb, at least about 70 ppb, at least about 80 ppb, at least about 90 ppb, at least about 100 ppb or anywhere in between of butanol.

In one embodiment, a subject who is likely to have hyperglycemia has at least about 2.1 ppb, at least about 2.2 ppb, at least about 2.3 ppb, at least about 2.4 ppb, at least about 2.5 ppb, at least about 2.6 ppb, at least about 2.7 ppb, at least about 2.8 ppb at least about 2.9 ppb, at least about 3 ppb, at least about 4 ppb, at least about 5 ppb, at least about 6 ppb, at least about 7 ppb, at least about 8 ppb, at least about 9 ppb, at least about 10 ppb, at least about 20 ppb, at least about 30 ppb, at least about 40 ppb, at least about 50 ppb, at least about 60 ppb, at least about 70 ppb, at least about 80 ppb, at least about 90 ppb, at least about 100 ppb or anywhere in between of pentanol.

The methods of the present invention can be used to determine whether a subject is likely or unlikely to have hyperglycemia. It may also be used to predict the likelihood of the subject having, or the likelihood of a subject developing, diabetes or a diabetic condition. This is done by determining the level of at least one of cymene, butanol and pentanol in a sample obtained from the subject and/or comparing the level of the one or more biomarkers to a reference.

Likelihood is suitably based on mathematical modeling. An increased likelihood, for example, may be relative or absolute and may be expressed qualitatively or quantitatively. For instance, an increased risk may be expressed as simply determining the subject's level of a given biomarker at one or more time points and placing the test subject in an “increased risk” category, as compared to a reference, for example, from previous population studies at the same time points. Alternatively, a numerical expression of the test subject's increased risk may be determined based upon biomarker level analysis.

In one embodiment, the method comprises comparing the level of the one or more biomarkers. The comparison of the levels of the biomarkers can be made in comparison to one another or to a reference. The comparison of the levels to one another may involve the determination of a ratio, wherein the value of the ratio as compared to a reference predicts the likelihood of hyperglycemia in the subject.

In one embodiment, likelihood is assessed by comparing the level or abundance of one or more biomarkers to one or more preselected level, also referred to herein as a threshold or reference levels. Thresholds may be selected that provide an acceptable ability to predict risk etc.

In one embodiment, the subject is considered to be likely to have hyperglycemia where at least one biomarker in the sample from the subject is upregulated or downregulated as compared to the corresponding biomarker in a healthy subject, as described herein.

The method as defined herein may comprise determining a weighted score based on the level of each biomarker in a panel of biomarkers and comparing it to a weighted score obtained from a reference or control sample. Alternatively, the weighted score on the level of each biomarker in the panel of biomarkers in the sample may be compared to a pre-determined value.

For any particular biomarker, a distribution of biomarker levels for subjects who are likely or unlikely to have hyperglycemia may overlap. Under such conditions, a test may not absolutely distinguish a subject who likely or unlikely to have hyperglycemia with absolute (i.e., 100%) accuracy, and the area of overlap indicates where the test cannot distinguish the two subjects. A threshold can be selected, above which (or below which, depending on how a biomarker changes with risk) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143:29-36 (1982)).

In one embodiment, the level of the one or more biomarkers may be weighted according to their respective coefficient in a logistic regression to derive an output or value.

In some embodiments, one or more biomarkers is used to assign a risk score which describes a mathematical equation for evaluation or prediction of risk. The evaluation of risk may also take into account genotype (including described HLA genes) and other clinical or phenotypic features, such as age.

In one embodiment, the method comprises detecting one or more risk factors in the subject. In one embodiment, the subject has been pre-selected based on one or more risk factors. The risk factors may be one or more risk factors as shown in Table 9.

The method as defined herein may take into account one or more of the following factors: a) weight, b) age, c) family history, d) physical lifestyle and/or activity, e) gender, f) race and ethnicity, g) history of major illness or injury, h) atmospheric or environmental VOCS and i) time before or after food before a sample is taken.

In some embodiments, the data sets corresponding to biomarker profiles are used to create a diagnostic or predictive rule or model based on the application of a statistical and machine learning algorithm. Such an algorithm uses relationships between a biomarker profile and the likelihood of hyperglycemia observed in control subjects or typically cohorts of control subjects (sometimes referred to as training data), which provides combined control or reference biomarker profiles for comparison with biomarker profiles of a subject. The data are used to infer relationships that are then used to predict the likelihood of hyperglycemia in a subject.

The method may comprise detecting hyperglycemia in the subject.

In one embodiment, the method predicts the likelihood of the subject having, or the likelihood of developing, diabetes or a diabetic condition. The diabetes may be type I diabetes mellitus or type II diabetes mellitus. The diabetic condition may be a condition due to type I diabetes mellitus or type II diabetes mellitus.

In one embodiment, the method comprises treating hyperglycemia in the subject found likely to have hyperglycemia.

The terms “subject,” “individual” and “patient” are used interchangeably herein to refer to any subject, particularly a vertebrate subject, and even more particularly a mammalian subject. Suitable vertebrate animals that fall within the scope of the invention include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc), marine mammals (e.g., dolphins, whales), reptiles (snakes, frogs, lizards, etc.), and fish. A preferred subject is a primate (e.g., a human, ape, monkey, chimpanzee). In one embodiment, the subject is a human.

In one embodiment, there is provided a method of predicting the likelihood of hyperglycemia in a subject, the method comprising comparing the level of one or more biomarkers in a sample obtained from the subject to a reference, wherein the level or levels of the one or more biomarkers, or a value derived therefrom, as compared to the reference predicts the likelihood of hyperglycemia in the subject; and wherein the one or more biomarkers comprises at least one of cymene, butanol and pentanol.

Provided herein is a kit for performing any of the methods as defined herein. The kit may be configured to determine the level of one or more biomarkers in a sample obtained from the subject; wherein the one or more biomarkers comprises at least one of cymene, butanol and pentanol.

Disclosed herein is a method of treating hyperglycemia in a subject, the method comprising: a) determining the level of one or more biomarkers in a sample obtained from the subject, wherein the level or levels of the one or more biomarkers, or a value derived therefrom, as compared to a reference predicts the likelihood of hyperglycemia in the subject; wherein the one or more biomarkers comprises at least one of cymene, butanol and pentanol, and b) exposing the subject to a treatment regimen to treat hyperglycemia in the subject.

The term “treatment regimen” encompasses natural substances and pharmaceutical agents (i.e., “drugs”) as well as any other treatment regimen including but not limited to dietary treatments, physical therapy or exercise regimens, surgical interventions, and combinations thereof. The treatment regimen to be adopted or prescribed may depend on several factors, including the age, weight and general health of the subject. The treatment regimen may also depend on existing clinical parameters, including body mass index, weight, glucose intolerance and homeostatic insulin resistance.

Thus, the present invention contemplates exposing the subject to a treatment regimen if the subject is determined to be likely to have hyperglycemia. The subject may, for example, be given exercise therapy, dietary treatment or medications such as metformin or insulin therapy. A subject found to have, or likely to develop, diabetes or a diabetic condition, will also be given the appropriate treatment.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing 5 a decrease in the severity of one or more symptoms of the disorder.

In one embodiment, there is provided a method of treating hyperglycemia in a subject, the method comprising:
a) comparing the level of one or more biomarkers in a sample obtained from the subject to a reference, wherein the level or levels of the one or more biomarkers, or a value derived therefrom, as compared to the reference predicts the likelihood of hyperglycemia in the subject; wherein the one or more biomarkers comprises at least one of cymene, butanol and pentanol, and b) exposing the subject to a treatment regimen to treat hyperglycemia in the subject.

Disclosed herein is a method of monitoring blood glucose level in a subject, the method comprising determining the level of one or more biomarkers in a sample obtained from the subject, wherein the level or levels of the one or more biomarkers, or a value derived therefrom, as compared to a reference provides an indication of the blood glucose level in the subject; and wherein the one or more biomarkers comprises at least one of cymene, butanol and pentanol.

Disclosed herein is a method of identifying a subject who is at risk of developing diabetes or a diabetic condition, the method comprising determining the level of one or more biomarkers in a sample obtained from the subject, wherein the level or levels of the one or more biomarkers, or a value derived therefrom, as compared to a reference identifies a subject as one who is at risk of developing diabetes or a diabetic condition and wherein the one or more biomarkers comprises at least one of cymene, butanol and pentanol.

Disclosed herein is a method of preventing or delaying the onset of diabetes or a diabetic condition in a subject, the method comprising a) determining the level of one or more biomarkers in a sample obtained from the subject, wherein the level or levels of the one or more biomarkers, or a value derived therefrom, as compared to a reference identifies a subject as one who is at risk of developing diabetes or a diabetic condition; wherein the one or more biomarkers comprises at least one of cymene, butanol and pentanol; and b) exposing the subject to a treatment regimen for preventing or delaying the onset of the diabetes or diabetic condition.

The articles “a” and “an” are used herein to refer to one or to more than one (i.e., to at least one) of the grammatical object of the article. By way of example, “a biomarker” means one biomarker or more than one biomarker.

As used herein, “and/or” refers to and encompasses any and all possible combinations of one or more of the associated listed items, as well as the lack of combinations when interpreted in the alternative (or).

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1

This report describes the pipeline of validating the selected biomarkers on hyperglycemia test by a machine learning approach. Specifically, a dataset of breath Volatile Organic Compounds (VOCs) was first collected and labelled with the last meal time before the data collection. Secondly, a machine model is constructed based on the selected VOC of the dataset. Finally, the overall result of built model on diet time diagnosis is shown along with the control experiments validating the effectiveness of the selected biomarkers.

### Machine Learning Pipeline

### Dataset Construction

The utilized dataset contains 335 samples collected at one clinical site. The sample analysis was conducted using a PTR-ToF-MS (Ionicon, PTR-TOF 4000). During data collections, each sample was asked to perform at least one complete respiratory cycle on the PTR-MS machine, and be recorded with the time after the last meal. Specifically, data of each sample contains: collection date, full PTR-MS spectra v.s. time, time since last meal, other recorded data of PTR-MS operation. The statistics of samples are shown in Table 3.

TABLE 3
Statistics of Collected Dataset

| Gender | | Age | Time Since Last Meal (hr) | | |
|---|---|---|---|---|---|
| Female | Male | Mean ± Std | within 3 | 3 to 6 | exceed 6 |
| 159 | 176 | 34.3 ± 15.4 | 163 | 72 | 100 |

### Data Processing

### Peak Extraction

Peaks are extracted from spectra data by peak detection algorithms in IONICON viewer software. It is noted that the intensity of extracted peaks is measured in ion counts, thus further transformations are performed on the intensity value. The transformations utilize PTR-transmission information and recorded reaction rate to transform the intensity into concentration (Unit: ppb). Overall, total 600 tracing peaks, i.e., VOCs, with their concentrations are extracted for each data sample.

### Data Annotation

The time since last meal is converted to a binary label (i.e., 0 and 1) by thresholding. The threshold is selected to be 3 hours, as human blood glucose level usually become normal within 3 hours after a meal. We here refer to the samples within 3 hours since meal as “potential high blood glucose” (PHBG) samples, and refer to the samples exceed 3 hours since meal as “potential normal blood glucose” (PNBG) samples.

### Train/Test Split

The dataset is randomly spitted by training/testing set with the ratio 70%/30%.

TABLE 4
Statistics of Dataset Train/Test Split

| Potential blood glucose level | # in Training Set | # in Testing Set |
|---|---|---|
| High | 117 | 46 |
| Normal | 118 | 54 |

### Model Description

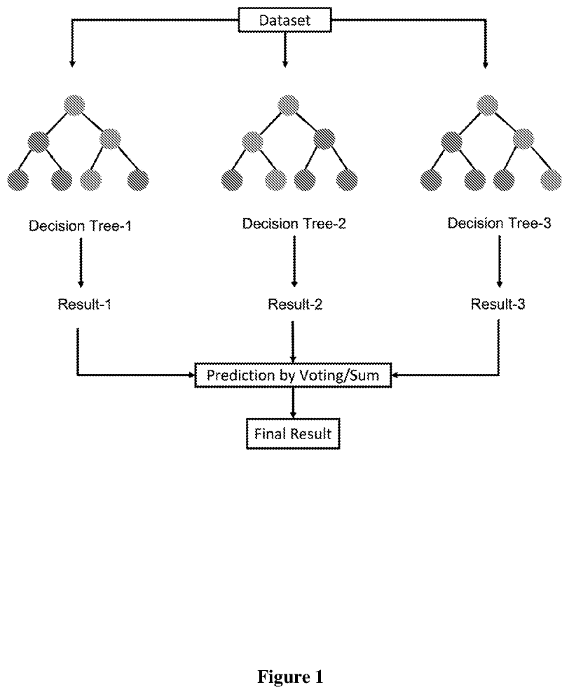

Based on the processed dataset, a machine learning model is built to diagnose potential blood glucose level by PTR-MS breath data. To address the possible over-fitting and non-normalization problem, the utilized machine learning model is selected as Random Forest. Random forest is an ensemble learning method for classification that operates by constructing a multitude of decision trees at training time. For classification tasks, the output of the random forest is the class selected by most trees. An illustration of random forest is displayed on FIG. 1.

Parameter Setting The random forest is implemented by Scikit-learn python package. The important parameter description and our customized setting is shown below:
criterion The function to measure the quality of a split in decision tree, which is set as ‘gini’.
max_depth The maximum depth of the tree, which is set 2 in our implementation.
n_estimators The number of base decision trees in the random forest, which is set as 32.
max_samples The number of samples to draw from training set to train each tree. The number is set as the 70% of training dataset.

### Model Result

After training process, the learned machine learning model performs classification on the testing set. In Table 5, we report the performance of the output model built based on our selected biomarkers (cymene, butanol, and pentanol) in comparison of models based on random VOCs group with the same number of VOCs.

TABLE 5
Comparison between Random VOCs Groups and Selected VOCs

| Group | Sensitivity(%) | Specificity(%) | Accuracy(%) |
|---|---|---|---|
| Random Group 1 | 50.0 | 58.7 | 54.3 |
| Random Group 2 | 42.6 | 69.6 | 56.1 |
| Random Group 3 | 25.9 | 84.8 | 55.4 |
| Random Group 4 | 0.0 | 100.0 | 50.0 |
| Random Group 5 | 1.9 | 97.8 | 49.8 |
| Random Group 6 | 74.1 | 28.3 | 51.2 |
| Random Group 7 | 46.3 | 50.0 | 48.1 |
| Random Group 8 | 40.7 | 56.5 | 48.6 |
| Random Group 9 | 66.7 | 26.1 | 46.4 |
| Random Group 10 | 51.9 | 56.5 | 54.2 |
| Random Group Average | 40.0 | 62.8 | 51.4 |
| Only Cymene VOCs | 77.8 | 76.1 | 77.0 |
| Only Butanol VOCs | 55.6 | 73.9 | 64.8 |
| Only Pentanol VOCs | 66.7 | 76.1 | 71.4 |
| Selected All Biomarkers | 88.9 | 85.8 | 86.8 |
| Group | | | |

Each of Random Groups 1-10 contains 7 VOCs that do not include the VOCs of Cymene (m/z at 135, 93 or 91), Butanol (m/z at 75 or 57) or Pentanol (m/z at 71 or 87) (see Table 7 below).

“Only Cymene VOCs” contain VOCs with m/z at 135, 93 and 91. “Only Butanol VOCs” contain VOCs at m/z at 75 and 57. “Only Pentanol VOCs” contain VOCs with m/z at 71 and 87.

“Selected All Biomarkers Group” contains VOCs for Cymene (m/z at 135, 93 and 91), Butanol (m/z at 75 and 57) and Pentanol (m/z at 71 and 87).

TABLE 6
VOCs in Random VOCs Group

| | group | group | group | group | group | group | group | group | group | group |
|---|---|---|---|---|---|---|---|---|---|---|
| | 1 | 2 | 3 | 4 | 5 | 6 | 7 | 8 | 9 | 10 |
| VOC 1 | 427.0 | 279.0 | 416.0 | 488.0 | 300.0 | 78.0 | 72.1 | 127.0 | 110.0 | 58.0 |
| VOC 2 | 223.0 | 73.0 | 289.0 | 436.0 | 58.0 | 445.0 | 215.0 | 355.0 | 101.0 | 319.0 |
| VOC 3 | 205.0 | 288.0 | 182.0 | 431.0 | 191.0 | 370.0 | 168.0 | 28.0 | 139.0 | 148.0 |
| VOC 4 | 447.0 | 240.0 | 122.0 | 256.0 | 494.0 | 144.0 | 98.0 | 63.0 | 17.0 | 415.0 |
| VOC 5 | 272.0 | 254.0 | 282.0 | 32.0 | 128.0 | 272.0 | 226.0 | 260.0 | 461.0 | 67.1 |
| VOC 6 | 83.1 | 50.0 | 45.0 | 211.0 | 112.0 | 199.0 | 340.0 | 103.9 | 244.0 | 244.0 |
| VOC 7 | 458.0 | 46.0 | 319.0 | 448.0 | 284.0 | 25.0 | 444.0 | 360.0 | 301.0 | 424.0 |

### Details of Selected VOCs

We calculated the importance for each input VOCs in the resulted model by averaging the information gains across all decision trees in the model. Table 7 summarized all used VOCs and their range in PHBG and PNBG samples, respectively.

TABLE 7
Selected VOCs and Result Analysis

| | Traced | Range at | Range at | |
|---|---|---|---|---|
| | VOC | PHBG | PNBG | Relative |
| Biomarkers (ion) | (m/z) | (ppb) | (ppb) | Importance |
| Cymene (fragment 1) | 135 | 0~0.6 | 0~0.5 | 0.17 |
| Cymene (fragment 2) | 93 | 0~2.3 | 0~2.0 | 0.16 |
| Cymene (fragment 3) | 91 | 0.7~3.4 | 0.4~3.0 | 0.11 |
| Butanol (fragment 1) | 75 | 0~5.2 | 0~2.6 | 0.08 |
| Butanol (fragment 2) | 57 | 0~3.7 | 0~3.2 | 0.01 |
| Pentanol (fragment 1) | 71 | 0.2~5.6 | 0.1~1.5 | 0.25 |
| Pentanol (fragment 2) | 87 | 0.1~0.9 | 0~0.8 | 0.22 |

### Example 2

The analysis of VOCs present in exhaled breath can provide valuable information about the subjects' physiological and pathophysiological conditions. Such compounds can be useful indicators and potential biomarkers of various diseases and metabolic activities, facilitating disease diagnosis. It should be noted that biological monitoring is generally based on the analysis of blood. However, this involves an invasive and time-consuming technique, which is often unacceptable to patients. The invasive techniques also require skilled medical staff. Breath analysis is an attractive alternative as it is a non-invasive and quick method that allows repeated sampling. In our study, we have identified 20 VOCs from exhaled breath for its association with blood glucose levels, HbA1c and cytokine induced inflammation of hyperglycemia patients. The list of 20 VOCs are divided into three levels based on their importance in associating with BGL (blood glucose level), HbA1c and the related inflammatory response. It is important to determine all VOCs concentration level changes within ppb level to pptv level or even at lower level of detection at ppm level using sensors that could be used to associate with blood glucose mmol/L level, Hba1c level and inflammatory response for prediabetes due to hyperglycemia.

The experimental findings obtained enable us to gain insights into possible screening and monitoring strategies for T2DM patients with hyperglycemia by looking for a possible correlation between BGL, Hba1c, even inflammatory response and the related breath VOCs level changes. The VOC concentrations can also be detected with a handheld sensor for non-invasive breath testing. Thus it is important to have differentiated level of associated VOCs for the detection of likelihood of hyperglycemia. Detection of hyperglycemia does not mean the subject has diabetes mellitus but it is important for subjects to monitor their occurrence of hyperglycemia through a non-invasive method.

The study involves using breath VOC concentration level change in ppb or pptv level using a highly sensitive Proton Transfer Reaction-Mass Spectrometry (PTR-MS) to identify the VOCs to detect the likelihood of hyperglycemia associated with BGL, h1Ac level and cytokines biomarkers provide novel knowledge. This can potentially contribute significantly towards managing T2DM in patients managing their BGL level in future by detecting their breath VOCs and managing their treatment.

Type 1 diabetes is characterized by a complete or near-complete insulin deficiency caused by an immune mediated selective destruction of the insulin-producing B-cells in the islets of Langerhans. Type 1 diabetes can be considered an inflammatory disease of the pancreatic islets in which a process of programmed cell death (apoptosis) is elicited in the B-cells by interaction of activated T-cells and proinflammatory cytokines in the immune infiltrate. The immune-mediated β-cell destruction is thought to be initiated by interaction between yet unknown environmental factors and type 1 diabetes susceptibility gene variants.

Type 2 diabetes is characterized by the failure of the B-cells to compensate for peripheral insulin resistance. Within the last decade, an increasing body of evidence has accumulated in favor of a putative role of immuno-related mechanisms and factors in the pathogenesis of type 2 diabetes, both with regard to the progressive β-cell failure and destruction and to the peripheral insulin resistance.

Interleukin (IL)-6 is a pleiotropic cytokine with a key impact on both immunoregulation and nonimmune events in most cell types and tissues outside the immune system. A vast number of epidemiological, genetic, rodent, and human in vivo and in vitro studies have investigated the putative role of action/lack of action of IL-6 in the pathogeneses underlying obesity, insulin resistance, β-cell destruction, type 1 diabetes, and type 2 diabetes. These studies suggest both protective and pathogenetic actions of IL-6 in diabetes.

Evidence continues to pile during the last two decades regarding the clinical relevance of laboratory predictors of pathophysiological events such as diabetes or hyperglycemia. These predictors are molecules, usually in the picogram (pg) range, called biomarkers. Inflammatory mechanisms play a key role in the pathogenesis of type 1 diabetes. Individuals who progress to type 2 diabetes display features of low-grade inflammation years in advance of disease onset. This low-grade inflammation has been proposed to be involved in the pathogenetic processes causing type 2 diabetes. Numerous studies have demonstrated that in individuals with impaired fasting blood glucose, circulating levels of inflammatory markers such as C-reactive protein (CRP), sialic acid and Interleukin-6 (IL-6) are independent predictors of the future development of diabetes. Furthermore high levels of circulating acute phase proteins in particular IL-6, tumor necrosis factor α (TNF-α) and other mediators of inflammation such as serum-amyloid A (S-AA), and CRP and their association with obesity and insulin resistance have been previously documented. Additionally several studies have demonstrated elevated levels of IL6 and TNF-α among individuals with insulin resistance and clinically diagnosed diabetes. An important factor that could potentially contribute to inflammation is chronic hyperglycemia.

Metabolic end products resulting from poorly controlled glucose are known to up-regulate the innate immune system leading to inflammation. In a recent report, IL-6 was found to be strongly associated with levels of glucose and was a strong predictor of diabetes in at-risk individuals. Inadequate glucose control and its associated inflammation in diabetes have been implicated in the pathogenesis of atherosclerosis, impaired lung function and cardiovascular disease.

In our detection, breath emitted VOCs such as p-cymene, 1-butanol, 1-pentanol are found to be strongly associated with IL-6 or interleukin families and more prominently tumor necrosis factor α (TNF-α) due to systemic inflammation are considered to be independent predictors of the future development of diabetes. This will be the level 1 factors (refer to Table 8) for the VOCs novelty for use to detect onset of diabetes. Subjects with no hyperglycemia, on the other hand, are found to have about 0.02 ppb to about 0.6 ppb P-cymene, about 0.5 ppb to about 5 ppb 1-butanol and/or about 0.1 ppb to about 2.1 ppb 1-pentanol in their breath.

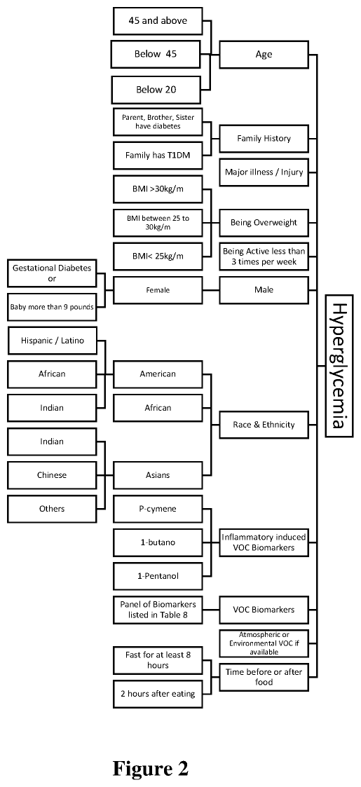

The elevated levels of these VOCs (p-cymene, 1-butanol, 1-pentanol) are strongly associated with subjects with mild inflammation. Although inflammation in the body could be caused by other possibly infections but these breath emitted VOCs (p-cymene, 1-buthanol, 1 penthanol) will be used with other sets of VOCs for detection of hyperglycemia. For example, the likelihood of hyperglycemia can be detected through breath VOCs (Table 8) and its associated inflammation in the body and qualitative criteria (risk factors-Table 9) as listed in the decision example in FIG. 2. The algorithm of each input or decision can be adjusted to improve the sensitivity and specificity (FIG. 2).

TABLE 8
Biomarker list from Breath Analysis

| | Detection | | |
|---|---|---|---|
| | Decision | | |
| | Priority | Biomarkers | Pathway or known possibly |
| No. | Level | (VOCs) | relation |
| 1 | Level 1 | p-cymene | Cytokine & Pathogenic processes |
| 2 | Level 1 | 1-butanol | Cytokine & Pathogenic processes |
| 3 | Level 1 | 1-pentanol | Cytokine & Pathogenic processes |
| 4 | Level 1 | Acetone* | Glycolysis/Pyruvate Metabolism |
| 1 | Level 2 | ethanol | Glycolysis/Gluconeogenesis |
| 2 | Level 2 | Propane | N-4 fatty acid peroxidation protein |
| | | | oxidation |
| 3 | Level 2 | Carbon monoxide | |
| 4 | Level 2 | Ethyl benzene | |
| 5 | Level 2 | M/P Xylene | |
| 6 | Level 2 | O-xylene | |
| 7 | Level 2 | methanol | |
| 8 | Level 2 | Isoprene | |
| 1 | Level 3 | Methyl Nitrate | |
| 2 | Level 3 | 2-pentyl nitrate | |
| 3 | Level 3 | Toulene | |
| 4 | Level 3 | Tridecane | |
| 5 | Level 3 | Undecane | |
| 6 | Level 3 | Trimethyldecane | |
| | | (2,7,8) | |
| 7 | Level 3 | Trimethyldecane | |
| | | (3,5,7) | |
| 8 | Level 3 | Pentane | |

TABLE 9
Risk factors for prediabetes or hyperglycemia

| | | Decision Input for |
|---|---|---|
| Risk | | determining the |
| Factor | | likelihood for |
| No. | Risk Factor | hyperglycemia |
| 1 | Overweight (BMI) | Being Overweight |
| 2 | Being 45 years or older | Age |
| 3 | Having family members (example a parent, brother, or sister) | Family History |
| | with type 2 diabetes | |
| 4 | Being physically active less than 3 times a week | Physical less active |
| 5 | Ever having gestational diabetes (diabetes during pregnancy) | Gender |
| | or giving birth to a baby who weighed more than 9 pounds | |
| 6 | Race and ethnicity are also a factor: African Americans, | Race and ethnicity |
| | Hispanic/Latino Americans, American Indians, and some | |
| | Asian Americans or Asians are at higher risk. | |
| 7 | There is also consideration of | Major Illness/ |
| | nondiabetic hyperglycemia means the blood glucose (sugar) | Injury |
| | level is high even though the subject have no | |
| | diabetes. Hyperglycemia may happen suddenly during a | |
| | major illness or injury. Instead, hyperglycemia may happen | |
| | over a longer period of time and be caused by a chronic | |
| | disease. | |
| 8 | Atmospheric or Environmental VOCs can cause interference | Atmospheric or |
| | and bodily response for inflammation which induces change | Environmental |
| | in breath VOCs | VOCS if available |
| 9 | Fast for at least 8 hours and 2 hours after food can induce | Time before or after |
| | interference to VOCs and BGL. | food |

Control of blood glucose is important for subjects with T2DM especially. More studies are in progress of associating the breath VOCs with C-reactive protein and sialic acid. This non-invasive real time method of using an end tidal breath with a PTR-TOF-MS for detection hyperglycemia can provide valuable data for machine learning using varying the decision tree for each risk factor input to achieve high sensitivity (95%) and high specificity (98%). Machine learning can be used to improve each varying factor.

Provided herein is therefore a method of detecting the presence of a hyperglycemia which may lead to diagnosis of T1DM or T2DM disease in a subject. The method comprising the step of detecting or measuring a panel of biomarkers. The term panel of biomarkers may refer to two or more biomarkers. The biomarkers are present in a sample obtained from a subject. The biomarkers may be volatile organic compounds or volatile organic compounds related to cytokine-induced biomarkers. The cytokine-induced breathe biomarkers are elevated levels of these (p-cymene, 1-butanol, 1-pentanol). The other breathe biomarkers comprise of Acetone, ethanol, propane, carbon monoxide, ethyl benzene, M/P Xylene, O-Xylene, methanol, isoprene, methyl nitrate, 2-pentyl nitrate, toluene, tridecane, undercane, Trimethyldecane (2,7,8), Trimethyldecane (3,5,7) and Pentane.

In summary, it is demonstrated that the use of a reliable and non-invasive method by direct blowing through an end tidal breath sampler into the mass spectrometry system (PTR-TOF-MS) comprising a list of volatile organic compound for detection hyperglycemia and later allow good management of T2DM for example. With VOCs sensors developed to detectable ppb or pptv levels, these list of VOCs can be uses as a decision tool or machine learning to detect hyperglycemia using a handheld tool. Because there has been an exponential increase of T2DM patients in Asia, good diabetes control is of utmost importance. This helps to decrease costs incurred to individuals and society as a whole downstream.

1. A method of predicting the likelihood of hyperglycemia in a subject, the method comprising determining the level of one or more biomarkers in a sample obtained from the subject, wherein the level or levels of the one or more biomarkers, or a value derived therefrom, as compared to a reference predicts the likelihood of hyperglycemia in the subject; and wherein the one or more biomarkers comprises at least one of cymene, butanol and pentanol.

2. The method of claim 1, wherein the sample is a breath sample.

3. The method of claim 1 or 2, wherein the method comprises comparing the level of the one or more biomarkers obtained from the subject to a reference.

4. The method of any one of claims 1 to 3, wherein the method comprises determining the levels of cymene, butanol and pentanol in the sample.

5. The method of any one of claims 1 to 4, wherein the method further comprises determining the level of one or more biomarkers listed in Table 1.

6. The method of any one of claims 1 to 5, wherein the method further comprises determining the level of at least one of acetone, ethanol, Propane, Carbon monoxide, Ethyl benzene, Xylene, methanol and Isoprene.

7. The method of any one of claims 1 to 6, wherein the method further comprises determining the level of at least one of Methyl Nitrate, Pentyl nitrate, Toulene, Tridecane, Undecane, Trimethyldecane and Pentane.

8. The method of any one of claims 1 to 7, wherein the method comprises detecting one or more risk factors in the subject.

9. The method of any one of claims 1 to 8, wherein the subject has been pre-selected for one or more risk factors.

10. The method of any one of claims 1 to 9, wherein the method comprises detecting hyperglycemia in the subject.

11. The method of any one of claims 1 to 10, wherein the method predicts the likelihood of the subject having, or the likelihood of developing, diabetes or a diabetic condition.

12. The method of any one of claims 1 to 11, wherein the method comprises treating hyperglycemia in the subject found likely to have hyperglycemia.

13. A kit for performing a method according to any one of claims 1 to 12.

14. A method of treating hyperglycemia in a subject, the method comprising:
a) determining the level of one or more biomarkers in a sample obtained from the subject, wherein the level or levels of the one or more biomarkers, or a value derived therefrom, as compared to a reference predicts the likelihood of hyperglycemia in the subject; wherein the one or more biomarkers comprises at least one of cymene, butanol and pentanol, and
b) exposing the subject to a treatment regimen to treat hyperglycemia in the subject.

15. A method of treating hyperglycemia in a subject, the method comprising:
a) selecting a subject who is likely to have hyperglycemia, wherein the level or levels of the one or more biomarkers, or a value derived therefrom, in a sample obtained from the subject as compared to a reference predicts that the subject is likely to have hyperglycemia, wherein the one or more biomarkers comprises at least one of cymene, butanol and pentanol, and
b) exposing the subject to a treatment regimen to treat hyperglycemia in the subject.

16. A method of monitoring blood glucose level in a subject, the method comprising determining the level of one or more biomarkers in a sample obtained from the subject, wherein the level or levels of the one or more biomarkers, or a value derived therefrom, as compared to a reference provides an indication of the blood glucose level in the subject, and wherein the one or more biomarkers comprises at least one of cymene, butanol and pentanol.

17. A method of identifying a subject who is at risk of developing diabetes or a diabetic condition, the method comprising determining the level of one or more biomarkers in a sample obtained from the subject, wherein the level or levels of the one or more biomarkers, or a value derived therefrom, as compared to a reference identifies the subject as one who is at risk of developing diabetes or a diabetic condition; and wherein the one or more biomarkers comprises at least one of cymene, butanol and pentanol.

18. A method of preventing or delaying the onset of diabetes or a diabetic condition in a subject, the method comprising a) determining the level of one or more biomarkers in a sample obtained from the subject, wherein the level or levels of the one or more biomarkers, or a value derived therefrom, as compared to a reference identifies a subject as one who is at risk of developing diabetes or a diabetic condition; wherein the one or more biomarkers comprises at least one of cymene, butanol and pentanol; and b) exposing the subject to a treatment regimen for preventing or delaying the onset of the diabetes or diabetic condition.
